(19) United States
(12) Patent Application Publication
(10) Pub. No.: US 2010/0280592 A1
(43) Pub. Date: Nov. 4, 2010

(54) DRAWSTRING FOR REMOVAL OF STENT

(76) Inventors: Kyong-Min Shin, Seoul (KR); Kang-Sun Hong, Seoul (KR)

Correspondence Address: IP & T Law Firm PLC, 7700 Little River Turnpike, Suite 207, Annandale, VA, 22003, US

(21) Appl. No.: 12/770,497

(22) Filed: Apr. 29, 2010

(30) Foreign Application Priority Data
Apr. 30, 2009 (KR) 10-2009-0037989

(51) Int. Cl. A61F 2/82

(52) U.S. Cl. 623/1.15

(57) ABSTRACT

Disclosed herein is a drawstring for removal of a stent. The drawstring includes a circumferential string which is made of soft material and is formed in an end of the stent body in an annular shape. A hook loop is formed by tying opposite ends of the circumferential string to each other. The hook loop protrudes a relatively long distance from the stent body. A marking wire made of radiopaque material is wound around the hook loop. Therefore, when X-rays or endoscope are applied to a relative portion of a patient's body, the position of the hook loop can be easily detected. Furthermore, because the hook loop is made of flexible and soft material, even though it comes into contact with an internal organ of the patient's body, the organ is prevented from being damaged by the hook loop.

### BACKGROUND OF THE INVENTION

1. Field of the Invention

The present invention relates generally to a drawstring for removing a stent from a patient's body and, more particularly, to a hook loop of a drawstring for removing a stent from a patient's body. The hook loop of the drawstring is made of soft material so that even though the drawstring comes into contact with the inner wall of a lumen of the patient's body, the inner wall of the lumen can be prevented from being damaged by the drawstring. Furthermore, the hook loop of the drawstring is configured such that the position thereof can be easily discerned when X-rays are applied to a relative portion of the patient's body.

2. Description of the Related Art

Generally, a medical stent is used to expand a lumen which has contracted in diameter or to prevent expanded lumens from contracting.

In other words, medical stents functions to expand a lumen which has contracted, for example, by the occurrence of a tumor such as cancer, or to prevent the progression of contraction of a lumen.

For instance, such a medical stent is used to cope with the contraction of a pancreatic duct because of acute pancreatitis, the contraction of a bile duct attributable to chronic cholangitis, the temporary contraction of a lumen occurring after surgery, etc. Particularly, the medical stent which has been used to prevent symptoms of the temporary contraction of a lumen or to expand a contracted lumen is removed from the lumen after the lumen has recovered its original state. In addition, when the stent is placed in the lumen at an incorrect position and thus does not function as normal or is rendered ineffectual, the stent which has been placed in the lumen must be removed therefrom before reoperation can be conducted.

Typically, the stent which can be removed is inserted into a lumen by a well-known catheter to prevent contraction of the lumen or expand the diameter of the lumen. When the stent is removed from the lumen, if a leading end of the stent with respect to the direction in which the stent is extracted can be contracted in diameter, the removal of the stent is facilitated.

Preferably, the stent is configured such that it can be removed from the lumen using a hook of a stent removal device. With regard to the structure for removal of the stent, a plurality of loops is integrally connected to a stent body and to a cover which covers the inner and outer surfaces of the stent body. In addition, at least one string is connected at both ends thereof to each loop.

Therefore, to remove the stent from the lumen, the hook is inserted into the stent body and moved forwards and backwards until it becomes hooked to one loop. Thereafter, the hook is pulled. Then, the stent contracts towards the central axis thereof.

When the hook is further pulled, the stent body is inserted into a tube of the stent removal device.

Subsequently, the stent is completely removed from the lumen by removing the tube and the hook from the lumen together.

A representative example of a conventional technique pertaining to such a stent was proposed in US Patent No. 2006/0190075 which is entitled ‘Structure of hook loop of drawstring for removal of stent’.

In this conventional technique, the stent includes a cylindrical stent body which is manufactured by weaving one or more strands of shape memory alloy wires or crossing them with each other in a zigzag manner such that a mesh is defined by the wires and bent portions are formed along the circumferences of both ends of the cylindrical stent body. The drawstring includes a circumferential string which is formed on one end of the cylindrical stent body into an annular shape in such a way that it is woven with the corresponding bent portions of the cylindrical stent body in a zigzag manner. The opposite ends of the circumferential string extend predetermined lengths from the cylindrical stent body. The hook loop is formed by tying the opposite ends of the circumferential string to each other.

To remove the stent which has been placed in a lumen, a user hooks a hook of a stent removal device to the hook loop and pulls it.

However, in the conventional technique, the drawstring is made of hard wire and the portion of the drawstring which protrudes from the stent is in the shape of a hook. Thus, after the stent is placed in an organ of a patient's body, when the organ moves and the stent moves along with the organ, the hook loop may come into contact with the inner wall of the organ and hurt the organ.

Furthermore, if conducting a process of discerning the stent to remove the stent from the organ includes applying X-rays to a relative portion of the patient's body, the stent is easily detected but it is not easy to discern the position of the hook loop.

In addition, because the hook loop is relatively short, it is difficult to place the hook loop in the lumen at an appropriate position where the hook of the stent removal device can be hooked to the hook loop. As well, the hook loop may become completely embedded in body tissues that have grown.

Therefore, a drawstring is required, which has a relatively long hook loop such that it can be easily placed in the lumen at an appropriate position, and which is flexible so as to prevent the inner wall of an organ from being damaged by the hook loop, and in which a marking means is applied to the hook loop such that when an X-ray or endoscope is applied to a relative portion of the patient's body, the hook loop can be easily detected.

### SUMMARY OF THE INVENTION

Accordingly, the present invention has been made keeping in mind the above problems occurring in the prior art, and an object of the present invention is to provide a drawstring for removal of a stent in which a hook loop protrudes a relatively long distance from a cylindrical stent body and is made of soft material which is flexible, so that even though the drawstring comes into contact with the inner wall of the lumen, the inner wall of a lumen is prevented from being damaged by the hook loop.

Another object of the present invention is to provide a drawstring in which a marking wire coated with radiopaque material is wound around the outer surface of the hook loop, so that when an operation of removing the stent from the lumen is conducted, the position of the stent in the lumen can be easily and precisely discerned by applying X-rays to the lumen.

A further object of the present invention is to provide a drawstring in which the marking wire is wound around the hook loop in a coiled manner, thus preventing it from disturbing movement of the hook loop.

Yet another object of the present invention is to provide a drawstring in which the hook loop is relatively long to prevent body tissues from completely embedding the hook loop after they have grown.

In order to accomplish the above object, in an aspect, the present invention provides a drawstring for removal of a stent, the stent having a cylindrical stent body formed by weaving at least one strand of a shape memory alloy wire such that a mesh is defined by the wire and bent portions are formed along circumferences of opposite ends of the stent body. The drawstring includes a circumferential string and a marking wire. The circumferential string is formed in an end of the stent body in an annular shape in such a way that the circumferential string passes through the corresponding bent portions of the stent body in a zigzag manner. A hook loop is formed by tying opposite ends of the circumferential string to each other. The hook loop protrudes a predetermined length from the stent body. The marking wire is wound around an outer surface of the hook loop and is made of radiopaque material. When an X-ray or endoscope is applied to a relative portion of a patient's body, an operation of discerning a position of the hook loop is facilitated.

In another aspect, the present invention provides a drawstring for removal of a stent, the stent having a cylindrical stent body formed by weaving at least one strand of a shape memory alloy wire such that a mesh is defined by the wire and bent portions are formed along circumferences of opposite ends of the stent body. A medical film is applied to an inner wall of the stent body. The drawstring includes a circumferential string and a marking wire. The circumferential string is formed in an end of the stent body in an annular shape in such a way that the circumferential string passes through the corresponding bent portions of the stent body and the medical film in a zigzag manner. A hook loop is formed by tying opposite ends of the circumferential string to each other. The hook loop protrudes a predetermined length from the stent body. The marking wire is wound around an outer surface of the hook loop and is made of radiopaque material. When an X-ray or endoscope is applied to a relative portion of a patient's body, an operation of discerning a position of the hook loop is facilitated.

As described above, in the present invention, a hook loop of a drawstring to remove a stent from a lumen of a patient's body protrudes a relatively long distance from a stent body. The drawstring is made of soft material which is flexible. Thus, even though the drawstring comes into contact with the inner wall of the lumen, it is prevented from being damaged by the hook loop.

Furthermore, a marking wire coated with radiopaque material is wound around the outer surface of the hook loop of the drawstring. Hence, when an operation conducted to remove the stent from the lumen is conducted, the position of the stent in the lumen can be easily and precisely discerned by applying X-rays to the lumen.

In addition, because the marking wire is wound around the hook loop in a coiled manner, it is prevented from disturbing movement of the hook loop.

Moreover, the hook loop of the drawstring is relatively long. Therefore, body tissues are prevented from completely embedding the hook loop after they have grown.

### BRIEF DESCRIPTION OF THE DRAWINGS

The above and other objects, features and advantages of the present invention will be more clearly understood from the following detailed description taken in conjunction with the accompanying drawings, in which:

FIG. 1 is of a perspective view of a stent and a perspective view showing a drawstring provided in the stent, according to an embodiment of the present invention;

FIG. 2 is a perspective view showing a marking wire wound around a hook loop of the drawstring provided in the stent of FIG. 1;

FIG. 3 is an enlarged view of a circled portion A of FIG. 2;

FIG. 4 is of sectional views taken along the line B-B of FIG. 2;

FIG. 5 is a perspective view showing a drawstring applied to a cylindrical stent having an inner surface to which a film is attached, according to another embodiment of the present invention;

FIG. 6 is a view showing insertion of the stent into a lumen; and

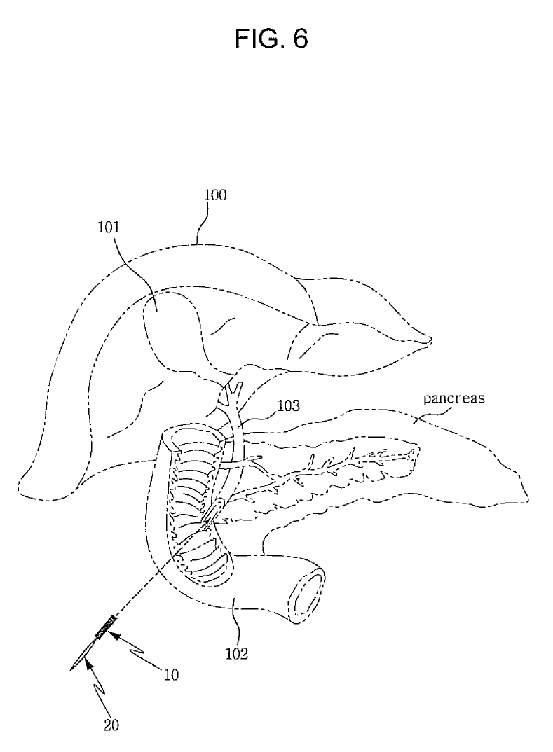

FIG. 7 is a view showing the stent placed in the lumen of FIG. 6.

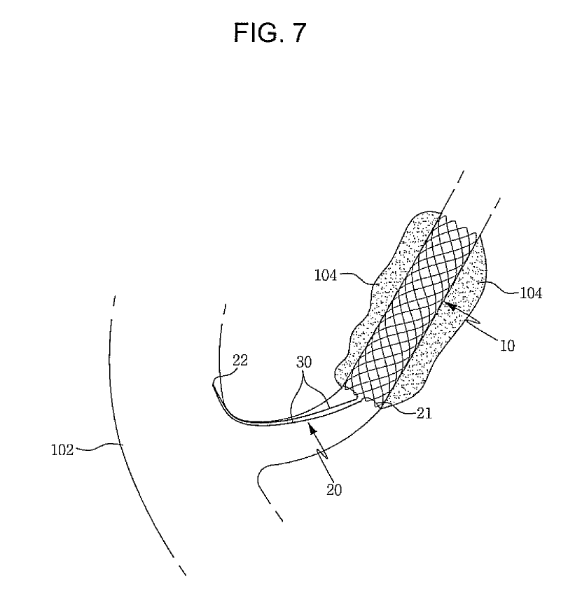

### DESCRIPTION OF THE PREFERRED EMBODIMENTS

Hereinafter, preferred embodiments of the present invention will be described in detail with reference to the attached drawings.

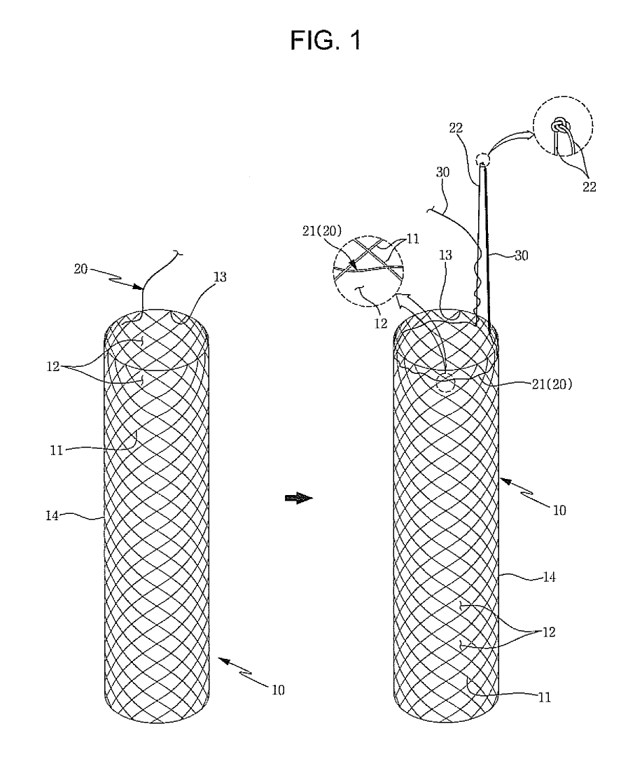

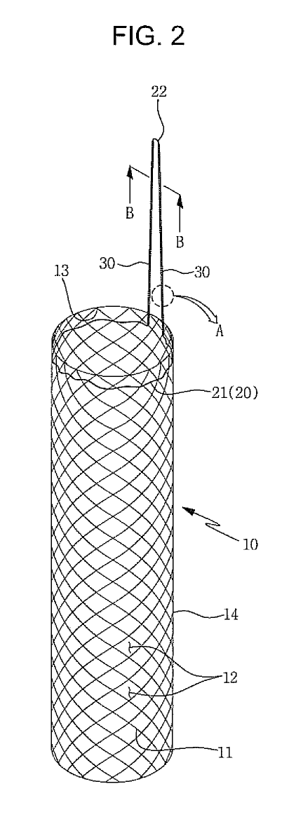

As shown in FIGS. 1 and 2, the present invention provides a structure of a hook loop 22 of a drawstring 20 for removal of a stent 10. The stent 10 includes a cylindrical stent body 14 which is manufactured by weaving one or more strands of shape memory alloy wires 11 or crossing them with each other in a zigzag manner such that mesh 12 is defined by the wires 11 and bent portions 13 are formed along the circumferences of both ends of the cylindrical stent body 14.

The drawstring 20 which functions to remove the stent 10 from a lumen of a patient includes a circumferential string 21 which is formed on one end of the cylindrical stent body 14 into an annular shape in such a way that it is woven with the corresponding bent portions 13 of the cylindrical stent body in a zigzag manner. The opposite ends of the circumferential string 21 extend relatively long distances from the cylindrical stent body 14. The hook loop 22 is formed by tying the opposite ends of the circumferential string 21 to each other.

Here, because the hook loop 22 extends a relatively long distance from the stent body 14, it can have high flexibility, the length thereof can be easily adjusted, and detection of the position of the hook loop 22 can be facilitated.

As such, the circumferential string 21 of the drawstring 20 is formed into an annular shape by weaving it with the bent portions 13 of the stent 10 in a zigzag manner. The hook loop 22 is formed by tying the opposite ends of the circumferential string 21 to each other.

The drawstring 20 is made of one selected from the group including synthetic resin and metal.

In addition, the drawstring 20 is preferably made of soft material which is flexible to prevent the lumen from being hurt by the drawstring 20 even when it comes into contact with the inner wall of the lumen.

In the present invention, a marking wire 30 is wound around the outer surface of the hook loop 22 in a coiled manner. The marking wire 30 is coated with radiopaque material.

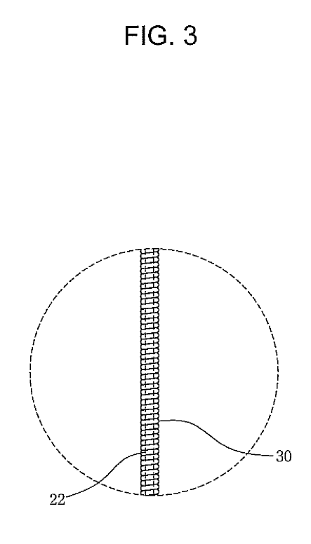

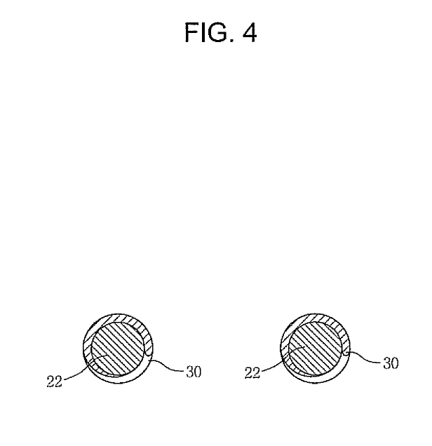

As shown in FIGS. 1, 3 and 4, the marking wire 30 is wound around the hook loop 22 of the drawstring 20 in the coiled manner such that the hook loop 22 can be easily bent.

Thanks to the marking wire 30, when X-rays or an endoscope are applied to a relative portion of the patient's body to remove the stent 10 from the lumen, the position of the hook loop 22 can be easily detected.

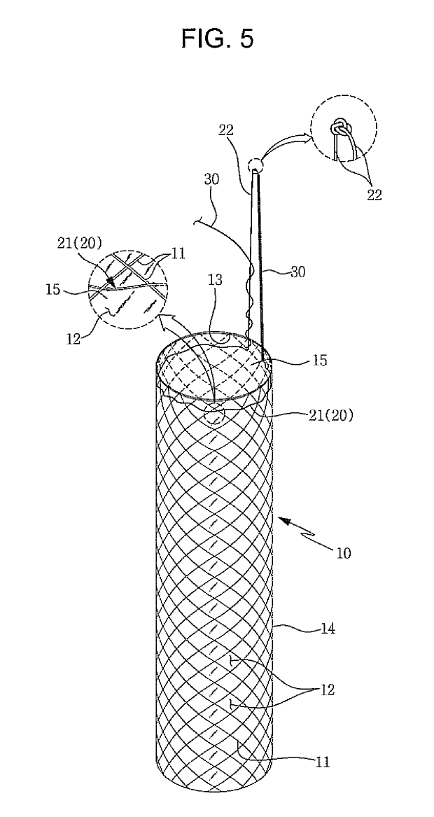

Meanwhile, FIG. 5 illustrates another embodiment of the present invention. As shown in FIG. 5, in this embodiment, a drawstring 20 is provided in a stent 10 to which a medical film 15 was applied. The stent 10 includes a cylindrical stent body 14 which is manufactured by weaving one or more strands of shape memory alloy wires 11 or crossing them with each other in a zigzag manner such that a mesh 12 is defined by the wires 11 and bent portions 13 are formed along the circumferences of both ends of the cylindrical stent body 14. The medical film 15 is attached to the inner wall of the cylindrical stent body 14.

In detail, the drawstring 20 includes a circumferential string 21 which is formed on one end of the cylindrical stent body 14 into an annular shape in such a way that it is woven with the corresponding bent portions 13 of the cylindrical stent body 14 in a zigzag manner. The opposite ends of the circumferential string 21 extend relatively long distances from the cylindrical stent body 14. A hook loop 22 is formed by tying the opposite ends of the circumferential string 21 to each other.

Here, because the hook loop 22 extends a relatively long distance from the stent body 14, the length thereof can be easily adjusted and detection of the position thereof can be facilitated.

As such, the circumferential string 21 of the drawstring 20 is formed into an annular shape by weaving it with the bent portions 13 of the stent 10 in a zigzag manner. The hook loop 22 is formed by tying the opposite ends of the circumferential string 21 to each other.

The drawstring 20 according to this embodiment is also made of one selected from the group including synthetic resin and metal.

In addition, the drawstring 20 is preferably made of soft material which is flexible so that it prevents the lumen of the patient from being damaged by the drawstring 20 even when it comes into contact with the inner wall of the lumen.

Furthermore, a marking wire 30 which is coated with radiopaque material is wound around the outer surface of the hook loop 22 in a coiled manner.

Because the marking wire 30 is wound around the hook loop 22 in the coiled manner, the hook loop 22 can be easily bent.

Thanks to the marking wire 30, when X-rays or an endoscope is applied to the portion of interest of the patient's body to remove the stent 10 from the lumen, the position of the hook loop 22 can be easily detected.

The operation and effect of the present invention having the above-mentioned construction will be explained.

As shown in FIGS. 1 through 5, the drawstring 20 is woven with the cylindrical stent body 14 into an annular shape in such a way that it is threaded with the corresponding bent portions 13 of the stent body 14 in a zigzag manner. Thereafter, the hook loop 22 is formed by tying both ends of the drawstring 20.

Subsequently, the marking wire 30 is densely wound around the entire portion of the hook loop 22 other than the tied end of the hook loop 22, thus completing formation of the drawstring 20.

Below the case where the stent 10 having the drawstring 20 is applied to a bile duct 103 through which bile of the liver 100 and the gall bladder 101 is supplied into a pancreatic duct 102 will be explained as one example.

To expand a portion with a lesion 104 of the bile duct 103, the stent 10 having the drawstring 20 is applied to the lesion portion 104 using a catheter which is a device to insert the stent into the lumen.

Here, the drawstring 20 which extends a relatively long distance from the end of the stent 10 is disposed such that the end of the drawstring 20 is placed in the pancreatic duct 102.

Even though the bile duct 103 or the pancreatic duct 102 moves due to the activity of the patient's body, the drawstring 20 is prevented from stimulating or damaging the inner wall of the organ, because the drawstring 20 is flexible and thus moves in response to the movement of the organ.

To remove the stent 10 from the bile duct 103 after the lesion has been completely recovered or when a restenting operation is required, a user hooks a hook of a stent removal device (not shown) to the hook loop 22 and then pulls it. Thereby, the stent 10 can be easily extracted from the bile duct 103.

In detail, when the hook is pulled after it is hooked to the hook loop 22, the stent 10 is compressed towards the diametrical center. When the hook is further pulled, the stent 10 is inserted into a tube of the stent removal device. Subsequently, the stent 10 is completely removed from the lumen by removing the tube and the hook from the lumen together.

Meanwhile, before undertaking such an operation of removing the stent 10 from the lumen, a process of detecting the stent 10 or the hook loop 22 must be conducted. For this, X-rays are applied to the patient's body or an endoscope is inserted into the pancreatic duct 102.

In the present invention, when X-rays are applied to the patient's body, the position and conditions of the hook loop 22 can be easily discerned by the radiopaque marking wire 20 wound around the hook loop 22.

Furthermore, when the endoscope is inserted into the lumen, the hook loop 22 which protrudes from the stent 10 can be easily located with the naked eye through a display or the position thereof can be easily discerned through the display because of the color of the radiopaque marking wire 30 wound around the hook loop 22.

As described above, in the present invention, since a hook loop is relatively long, a process of discerning the position of a stent to remove the stent from a lumen can be facilitated.

Furthermore, a marking wire coated with radiopaque material is wound around the hook loop. Thus, the position of the hook loop can be easily discerned in such a way as to apply X-ray to the patient's body.

In addition, because the radiopaque marking wire is wound around the hook loop, when an endoscope is used, the hook loop becomes highly visible.

As well, the drawstring is made of flexible material. Hence, even though the drawstring moves in the lumen, it is prevented from stimulating or damaging the inner wall of the lumen.

Moreover, in the present invention, because the hook loop of the drawstring is relatively long, it is prevented from being completely embedded with body tissues that are grown.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A drawstring for removal of a stent, the stent having a cylindrical stent body formed by weaving at least one strand of a shape memory alloy wire such that a mesh is defined by the wire and bent portions are formed along circumferences of opposite ends of the stent body, the drawstring comprising:
a circumferential string formed in an end of the stent body in an annular shape in such a way that the circumferential string passes through the corresponding bent portions of the stent body in a zigzag manner, with a hook loop formed by tying opposite ends of the circumferential string to each other, the hook loop protruding a predetermined length from the stent body; and
a marking wire wound around an outer surface of the hook loop, the marking wire being made of radiopaque material,
whereby when an X-ray or endoscope is applied to a relative portion of a patient's body, an operation of discerning a position of the hook loop is facilitated.

2. The drawstring as set forth in claim 1, which is made of one of a synthetic resin and a metal.

3. The drawstring as set forth in claim 1, which is made of flexible soft material to prevent an organ of the patient's body from being damaged by the drawstring when the drawstring comes into contact with the organ.

4. The drawstring as set forth in claim 1, wherein the marking wire is wound around the hook loop in a coiled manner such that the hook loop is bendable.

5. A drawstring for removal of a stent, the stent having a cylindrical stent body formed by weaving at least one strand of a shape memory alloy wire such that a mesh is defined by the wire and bent portions are formed along circumferences of opposite ends of the stent body, with a medical film applied to an inner wall of the stent body, the drawstring comprising:
a circumferential string formed in an end of the stent body in an annular shape in such a way that the circumferential string passes through the corresponding bent portions of the stent body and the medical film in a zigzag manner, with a hook loop formed by tying opposite ends of the circumferential string to each other, the hook loop protruding a predetermined length from the stent body; and
a marking wire wound around an outer surface of the hook loop, the marking wire being made of radiopaque material,
whereby when an X-ray or endoscope is applied to a relative portion of a patient's body, an operation of discerning a position of the hook loop is facilitated.

6. The drawstring as set forth in claim 5, which is made of one of a synthetic resin and a metal.

7. The drawstring as set forth in claim 5, which is made of flexible soft material to prevent an organ of the patient's body from being hurt by the drawstring when the drawstring comes into contact with the organ.

8. The drawstring as set forth in claim 5, wherein the marking wire is wound around the hook loop in a coiled manner such that the hook loop is bendable.

9. The drawstring as set forth in claim 2, which is made of flexible soft material to prevent an organ of the patient's body from being damaged by the drawstring when the drawstring comes into contact with the organ.

10. The drawstring as set forth in claim 6, which is made of flexible soft material to prevent an organ of the patient's body from being hurt by the drawstring when the drawstring comes into contact with the organ.
